(19) United States
(12) Patent Application Publication
(10) Pub. No.: US 2009/0220462 A1
(43) Pub. Date: Sep. 3, 2009

(54) STEM CELLS

(75) Inventor: John P. Chute, Durham, NC (US)

Correspondence Address: NIXON & VANDERHYE, PC, 901 NORTH GLEBE ROAD, 11TH FLOOR, ARLINGTON, VA, 22203, US

(73) Assignee: Duke University, Durham, NC (US)

(21) Appl. No.: 12/066,975

(22) PCT Filed: Sep. 14, 2006
(86) PCT No.: PCT/US06/35862

§ 371 Date: Jan. 13, 2009

Related U.S. Application Data
(60) Provisional application No. 60/716,501, filed on Sep. 14, 2005.

(51) Int. Cl. A61K 35/48; C12N 5/08; C12Q 1/02; A61K 35/44

(52) U.S. Cl. 424/93.7; 435/377; 435/29

(57) ABSTRACT

The present invention relates, in general, to stem cells and, in particular, to a method of expanding stem cells by inhibiting aldehyde dehydrogenase (ALDH). The invention further relates to methods of identifying compounds suitable for use in effecting expansion of stem cells.

This application claims priority from U.S. Provisional Application No. 60/716,501, filed Sep. 14, 2005, the entire content of which is incorporated herein by reference.

### TECHNICAL FIELD

The present invention relates, in general, to stem cells and, in particular, to a method of expanding stem cells by inhibiting aldehyde dehydrogenase (ALDH). The invention further relates to methods of identifying compounds suitable for use in effecting expansion of stem cells.

### BACKGROUND

Hematopoietic stem cells (HSCs) possess the unique capacity to self-renew and give rise to all mature lymphohematopoietic progeny throughout the lifetime of an individual (Osawa et al, Science 273:242-245 (1996), Sorrentino, Nat. Rev. Immunol. 4:878-888 (2004)). Several molecular pathways that regulate HSC self-renewal have now been identified, including Notch (Varnum-Finney et al, Nat. Med. 6:1278-1281 (2000)), HOXB4 (Krosl et al, Nat. Med. 9:1428-1432 (2003)), Wnt (Reya et al, Nature 423:409-414 (2003)) and bone morphogenetic protein signaling pathways (Bhardwaj et al, Nat. Immunol. 2:172-180 (2001)). The osteoblastic niche for HSCs within the bone marrow (BM) has also been characterized (Calvi et al, Nature 425:841-846 (2003), Zhang et al, Nature 425:836-841 (2003)). Despite these advances in understanding HSC biology, clinical methods to amplify human HSCs have yet to be realized and characterization of the pathways that regulate HSC self-renewal continues to evolve.

Two decades ago, Colvin et al. demonstrated that the intracellular enzyme, ALDH, protected bone marrow (BM) progenitors from the cytotoxic effects of cyclophosphamide, by deactivation of its metabolite, 4-hydroxycyclophosphamide (Colvin et al, Adv. Enzyme Regul 27:211-221 (1988), Russo et al, Prog. Clin. Biol. Res. 290:65-79 (1989)). Several isoforms of ALDH have been identified, with ALDH-1 being the primary isoform expressed within human hematopoietic progenitors (Kastan et al, Blood 75:1947-1950 (1990), Magni et al, Blood 87:1097-1103 (1996)). Recent studies have shown that human and murine hematopoietic progenitors can be isolated using a fluorescently-labeled dye specific for ALDH activity (Storms et al, Proc. Natl. Acad. Sci. USA 96:9118-9123 (2000), Jones et al, Blood 85:2742-2746 (1995), Hess et al, Blood 104:1648-1655 (2004), Storms et al, Blood 106:95-102 (2005)) and cord blood (CB) ALDH^{br}lin⁻ cells are enriched for nonobese diabetic/severe combined immunodeficiency (NOD/SCID) mouse repopulating cells (SRCs) (Hess et al, Blood 104:1648-1655 (2004), Storms et al, Blood 106:95-102 (2005)). While these data demonstrate that ALDH is a selectable marker for human stem/progenitor cells, the HSC-specific function of ALDH remains unknown. In the liver, ALDH-1 contributes primarily to the metabolism of retinol (Vitamin A) into retinoic acid (Bhat et al, Biochem. Pharmacol. 57:195-197 (1999)) and ALDH-1 is also concentrated in HSCs.

All-trans retinoic acid (ATRA), a derivative of Vitamin A, induces cellular differentiation, tissue patterning and embryonic development in vertebrates (Chambon, FASEB J. 10:940-954 (1996), Collins, Leukemia 16:1896-1905 (2002), Zechel, Mol. Endocrinol. 19:1629-1645 (2005), Zile, J. Nutr. 131:705-708 (2001)). Treatment of myeloid progenitors with ATRA induces terminal granulocytic differentiation (Collins, Leukemia 16:1896-1905 (2002), Tocci et al, Blood 88:2878-2888 (1996)) and ATRA is used therapeutically to induce the differentiation of acute promyelocytic leukemia cells, in which the characteristic 15; 17 translocation results in a fusion protein (PML-RARα) which harbors dominant negative activity against the RARα receptor (Tallman et al, N. Engl. J. Med. 337:1021-1028 (1997)). It has also been observed that the fold expansion of human cord blood (CB) and BM SRCs in co-culture with vascular endothelial cells correlates linearly with the amplification of CD34⁺ cells lacking expression of CD38, which is dependent on RARα signaling (Mehta et al, Blood 89:3607-3614 (1997), Chute et al, Blood 100:4433-4439 (2002), Chute et al, Blood 105:576-583 (2005)).

The present invention results, at least in part, from studies designed to test the hypothesis that inhibition of ALDH, which is required for the production of retinoic acids, could interfere with HSC differentiation. The studies described herein demonstrate that ALDH activity is necessary for normal HSC differentiation to occur in response to cytokines and that inhibition of ALDH, coupled with early acting cytokines, is sufficient to induce the quantitative expansion of human SRCs. These findings indicate that selective modulation of retinoid signaling via inhibition of ALDH can impart a robust expansion of HSCs.

### SUMMARY OF THE INVENTION

The invention relates generally to stem cells. More specifically, the invention relates to a method of expanding stem cells by inhibiting ALDH. The invention further relates to methods of identifying compounds suitable for use in effecting expansion of HSCs.

Objects and advantages of the present invention will be clear from the description that follows.

### BRIEF DESCRIPTION OF THE DRAWINGS

FIGS. 1A-1E. ALDH inhibition impedes the differentiation of human HSCs. FACS-sorted CB and BM CD34⁺CD38⁻lin⁻ cells were analyzed for ALDH activity levels and compared with the ALDH activity level in the CD34⁺CD38⁻ progeny of 7 day cultures with thrombopoietin, stem cell factor (SCF) and Flt3 ligand (TSF) alone versus TSF+DEAB (FIG. 1A). The surface expression of CD34 and CD38 on the day 7 progeny of CB CD34⁺CD38⁻lin⁻ cells cultured with TSF alone versus TSF+DEAB is shown (FIG. 1B) and identical analysis of CD34 and CD38 expression is shown for BM cells under the same culture conditions (FIG. 1C). DEAB+TSF maintained CD34⁺CD38⁻ cell numbers compared to input, whereas TSF alone caused a significant loss in CD34⁺CD38⁻ cells over time (FIG. 1D). Culture with TSF alone caused a marked increase in colony forming cell (CFC) content compared to input, whereas DEAB+TSF cultured progeny contained little CFC activity, indicating an inhibition of HSC differentiation during culture (FIG. 1E).

FIGS. 2A-2C. DEAB treatment promotes the amplification of human SRCs. (FIG. 2A) Representative flow cytometric analysis of human CD45 versus murine CD45 surface staining in NOD/SCID mice is shown at week 8 in mice transplanted with 1×10³ CB CD34⁺CD38⁻lin⁻ cells (top), their TSF-cultured progeny (middle) or their progeny following DEAB+TSF culture. (FIG. 2B) A scatter plot of human CD45+ cell engraftment in NOD/SCID mice at 8 weeks post-transplantation is shown with each individual point representing a single transplanted mouse. Mice transplanted with the progeny of CB CD34⁺CD38⁻lin⁻ cells cultured with DEAB+TSF demonstrated significantly increased frequency of human engraftment (>0.1%) and percent huCD45⁺ cell repopulation compared to day 0 CB CD34⁺CD38⁻lin⁻ cells or their progeny following culture with TSF alone. The mean levels of huCD45⁺ cells per culture condition are indicated by horizontal lines. (FIG. 2C) Multilineage engraftment of CD45⁺ cells, CD34⁺ progenitor cells, CD19⁺ B cells and CD 33/13⁺ myeloid cells is shown in the BM of a representative NOD/SCID mouse transplanted with the progeny of 2×10³ CB CD34⁺CD38⁻lin⁻ cells following DEAB+TSF culture, demonstrating normal in vivo differentiation of DEAB treated cells.

FIGS. 3A-3B. Treatment with ATRA accelerates the differentiation of primary human HSCs. FACS-sorted CB CD34⁺CD38⁻lin⁻ cells were cultured with TSF alone versus TSF+ATRA×7 days. (FIG. 3A) CD34 and CD38 surface expression is shown on day 0 cells (top), their progeny following TSF culture (middle) and their progeny following TSF+ATRA×7 days (bottom). ATRA induced a marked loss of CD34+ cells and CD34+CD38− cells in culture as compared to input or TSF culture, consistent with differentiation during culture. (FIG. 3B) The progeny of TSF+ATRA cultures also contained significantly less CFCs as compared to TSF cultured progeny, suggesting that ATRA induced the terminal differentiation or apoptosis of stem and progenitor cells in culture.

FIG. 4. Neither 5×10³ day 0 BM CD34⁺CD38⁻lin⁻ cells nor their progeny following TSF culture demonstrated long-term engraftment in any NOD/SCID mice, but the progeny of the identical dose of BM CD34⁺CD38⁻lin⁻ cells following culture with DEAB+TSF demonstrated human repopulation in 50-80% of transplanted mice.

FIGS. 5A and 5B. Treatment with DEAB maintains HOXB4 expression in HSCs. RNA was isolated from multiple replicates of FACS-sorted CB CD34⁺CD38⁻lin⁻ cells at day 0 and their progeny following culture with TSF alone or DEAB+TSF. The RNA was reverse transcribed and the expression of HOXB4 and Notch 1 was analyzed by quantitative real-time PCR. (FIG. 5A) The expression of HOXB4 in CD34⁺CD38⁻ cells was significantly reduced following short term culture with TSF, whereas treatment with DEAB prevented the downregulation of HOXB4 expression over time. (FIG. 5B) The expression of Notch was also significantly reduced following TSF culture but DEAB treatment did not altered this decline in Notch expression over time.

FIG. 6. When CB CD34⁺CD38⁻lin⁻ cells were cultured with 1 μM ATRA+100 μM DEAB+TSF, HSC differentiation occurred, indicating that provision of extracellular retinoids overcame DEAB-induced inhibition of intracellular retinoid production.

### DETAILED DESCRIPTION OF THE INVENTION

The present invention relates to a method of promoting expansion of stem cells, or progenitor cells, while inhibiting differentiation of such cells. The method comprising contacting the cells, for example, human hematopoietic stem cells (HSCs), with an inhibitor of ALDH under conditions such that expansion is effected.

Stem cells suitable for expansion in accordance with the invention include, for example, HSCs, neuronal stem cells and muscle stem cells. HSCs suitable for expansion can be obtained, for example, from bone marrow, umbilical cord blood or peripheral blood. Stem cells suitable for use can be separated from mixed populations of cells and cultured in the presence of ALDH inhibitor. The thus cultured cells can then be harvested.

Stem cells can be distinguished from most other cells by the presence or absence of particular antigenic marker antigens, such as CD34, that are present on the surface of these cells and/or by morphological characteristics. One phenotype of a highly enriched human stem cell fraction has been reported as CD34⁺, Thy-1⁺ and Lin⁻, however, the present invention is not limited to the expansion of this stem cell population.

A CD34⁺ enriched human stem cell fraction can be separated by a number of art-recognized techniques, including affinity columns or beads, magnetic beads or flow cytometry using antibodies directed to surface antigenics such as CD34⁺. The CD34⁺ progenitors can be divided into subpopulations characterized by the presence or absence of coexpression of different lineage-associated cell surface associated molecules. Immature progenitor cells do not express lineage associated markers such as CD38.

The separated cells can be incubated in a selected culture medium, for example, in a culture dish or flask, a sterile bag or hollow fiber. Various growth factors, e.g., hematopoietic growth factors, can be utilized in order to selectively expand cells. Representative factors include thrombopoietin, SCF and flt-3 ligand, or combinations thereof. Proliferation of the stem cells can be monitored by counting the number of stem cells using standard techniques (e.g., hemacytometer) or by flow cytometry prior and subsequent to incubations.

ALDH inhibitors suitable for use in accordance of the invention include, for example DEAB and metabolites thereof, as well as other known inhibitors such as those described in U.S. Pat. Nos. 5,624,910 and 6,255,497. The invention also includes methods of identifying ALDH inhibitors appropriate for use in effecting stem cell expansion. Candidate compounds can be screened for their ability to inhibit ALDH, particularly, ALDH-1 (e.g., specifically the ALDH-1 mediated metabolism of retinol to retinoic acid)), for their ability to block cytokine-induced differentiation of stem cells (e.g., HSCs) and/or for their ability to block stem cell differentiation by modulating HOXB4 expression/activity. The invention does not include the use of AGN 194310 (Prus et al, Leuk. Lymph. 45:1025 (2004)) in expanding stem cells or subpopulations thereof.

The ALDH inhibitors of the invention, advantageously used in combination with TSF (or other appropriate cytokine combination), result in the amplification of pluripotent cells that maintain normal differentiation capacity.

Stem cells expanded ex-vivo using an ALDH inhibitor of the invention can be used in the treatment of various diseases, including those characterized by decreased levels of either myeloid, erythroid, lymphoid or megakaryocyte cells of the hematopoietic system. In addition, they can be used to cultivate mature myeloid and/or lymphoid cells. Among conditions susceptible to treatment with hematopoietic cells expanded in accordance with the invention is leucopenia induced, for example, by exposure to viruses or radiation, or as a side effect of cancer therapy. The expanded cells of the invention can also be useful in preventing or treating bone marrow suppression or hematopoietic deficiencies that occur in patients treated with a variety of drugs.

The dosage regimen involved in ex vivo expansion of stem cells and methods for treating the above-described conditions can be determined by one skilled in the art and can vary with the ALDH inhibitor, the patient and the effect sought.

In addition to the ex vivo expansion of stem cells for therapeutic purposes (i.e., cord blood transplantation) the ALDH inhibitors can be used as systemic therapeutics, for example, for treating patients undergoing chemotherapy and/or radiotherapy to accelerate their hematopoietic recovery, as well as other patients suffering from blood cell disorders/deficiencies, including anemias (e.g., sickle cell anemia).

Certain aspects of the invention can be described in greater detail in the non-limiting Example that follows.

### Example

### Experimental Details

Isolation of Human BM and CB CD34⁺CD38⁻lin− Cells

Whole BM and CB units were obtained from the Duke University Stem Cell Laboratory within 48 hours of collection. Volume reduction of CB units was accomplished by 10 minute incubation at room temperature with 1% Hetastarch (Abbott Laboratories, North Chicago, Ill.), followed by centrifugation at 700 rpm for 10 minutes to facilitate component separation. The buffy coat was collected and washed twice with Dulbecco's phosphate buffered saline (PBS; Invitrogen, Carlsbad, Calif.) containing 10% heat-inactivated fetal bovine serum (FBS; Hyclone, Logan, Utah), 100 U/ml penicillin, and 100 μg/ml streptomycin (1% pcn/strp; Invitrogen). Cell pellets were resuspended in PBS with 10% FBS and 1% pcn/strp and overlaid onto Lymphoprep (Axis-Shield, Olso, Norway) and centrifuged at 1500 rpm for 30 minutes to isolate the mononuclear cell (MNC) fraction. MNCs were collected and washed twice before proceeding to lineage depletion.

Lineage depletion was conducted using the Human Progenitor Enrichment Cocktail (Stem Cell Technologies, Vancouver, Canada) which contains monoclonal antibodies (mAbs) to human CD2, CD3, CD14, CD16, CD9, CD56, CD66b, and Glycophorin A, according to the manufacturer's suggested protocol. Briefly, CB or BM MNCs were resuspended at 5-8×10⁷ cells/ml in PBS with 10% FBS and 1% pcn/strp and incubated with 100 μl/ml antibody cocktail for 30 minutes followed by incubation with 60 μl/ml magnetic colloid for 30 minutes. Cells were then magnetically depleted on a pump fed negative selection column (Stem Cell Technologies) using the manufacturer's recommended procedure. Lin⁻ cells were washed twice, quantified by hemacytometer count and cryopreserved in 90% FBS and 10% dimethylsulfoxide (DMSO; Sigma-Aldrich, St. Louis, Mo.) or utilized directly for further experiments.

Lin⁻ CB or BM cells were thawed, washed once in Iscove's Modified Dulbecco's Medium (IMDM; Invitrogen) containing 10% FBS and 1% pcn/strp, counted, and resuspended at 5-10×10⁷/ml. Immunofluorescent staining was conducted using anti-human CD34-fluorescein isothiocyanate (FITC) and anti-human CD38-phycoerythrin (PE) monoclonal antibodies (Becton Dickinson, San Jose, Calif.), for 30 minutes on ice. Stained cells were washed twice and resuspended at 1×10⁷ cells/ml in IMDM with 10% FBS and 1% pcn/strp. Analysis and sterile cell sorting was conducted using a FACSvantage flow cytometer (Becton Dickinson) to isolate CD34⁺CD38⁻ and CD34⁺CD38⁺ subsets. The CD34⁺CD38⁻ sort gate was set to collect only those events falling in the lowest 5% of PE fluorescence within the total CD34⁺ population, to ensure acquisition of highly purified CD34⁺CD38⁻ cells.

Analysis of In Vitro Hematopoietic Activity of Human CD34⁺CD38⁻lin⁻ Cells Following Culture with DEAB.

Primary human BM and CB CD34⁺CD38⁻lin⁻ cells were placed in culture with 20 ng/mL thrombopoietin, 100 ng/mL SCF, and flt-3 ligand (TSF), a cytokine combination previously found to induce human stem and progenitor cell proliferation and differentiation in vitro (Chute et al, Blood 105:576-583 (2005), Chute et al, Stem ells 22:202-215 (2004)). In order to assess whether inhibition of ALDH activity could block the differentiation of human HSCs induced to proliferate by TSF, 1-2×10⁴ BM or CB CD34⁺CD38⁻lin⁻ cells were cultured×7 days with IMDM, 10% FBS and 1% pcn/strp supplemented with TSF with and without 100 μM DEAB. At day 7, the progeny of all cultures were collected, washed×2 and cell counts were performed. A minimum of n=5 experiments was performed per culture condition to allow statistical comparisons to be performed. Immunophenotypic analysis was performed on progeny cells using anti-human CD34 and CD38 mAbs (Becton Dickinson) and isotype control IgG mAbs and compared with day 0 (input) staining. Fourteen day methylcellulose CFC assays (colony forming unit-granulocyte monocyte, CFU-GM, burst forming unit-erythroid, BFU-E, and colony forming unit-mix, CFU-Mix) were performed as we have previously described (Chute et al, Blood 100:4433-4439 (2002), Chute et al, Blood 105:576-583 (2005), Chute et al, Stem ells 22:202-215 (2004)) to compare the number of lineage committed CFCs within day 0 CD34⁺CD38⁻lin⁻ cells and their progeny. Morphologic analysis of day 0 CD34⁺CD38⁻lin⁻ cells and their progeny following 7 day culture was performed using Wrights-Geimsa staining and microscopic analysis under oil immersion (100×).

In order to assess the hematopoietic effects of retinoid agonists on purified human HSCs, BM and CB CD34⁺CD38⁻lin⁻ cells were placed in culture with TSF with and without 1 μM all-trans retinoic acid (ATRA), an agonist of RAR. Total cell expansion, immunophenotype, CFC content and morphologic analysis was performed on ATRA-treated progeny and compared with both day 0 CD34⁺CD38⁻lin⁻ populations and the progeny of TSF alone to assess the impact of retinoid agonism on the differentiation of human HSCs.

### In Vivo Long-Term Repopulating Assays in NOD/SCID Mice

NOD/SCID mice (Schulz et al, J. Immunol. 154:180-191 (1995)) were transplanted with either day 0 FACS-sorted BM or CB CD34⁺CD38⁻lin− HSC-enriched cells or the progeny of CD34⁺CD38⁻lin− cells cultured with TSF alone or TSF supplemented with 100 μM DEAB. Cells were transplanted via tail vein injection after irradiating NOD/SCID mice with 300 cGy using an X-ray irradiator as previously described (Chute et al, Blood 100:4433-4439 (2002), Chute et al, Blood 105:576-583 (2005)). Day 0 CD34⁺CD38⁻lin− HSCs and their progeny were co-transplanted with 2×10⁴ CD34⁺CD38⁻lin⁻ accessory cells to facilitate engraftment as previously described (Bonnet et al, Bone Marrow Transpl. 23:203-209 (1999), Bhatia et al, Proc. Natl. Acad. Sci. USA 94:5320-5325 (1997)).

All mice in each group were sacrificed at week 8 and marrow samples were obtained by flushing their femurs with IMDM at 4° C. Red cells were lysed using red cell lysis buffer (Sigma-Aldrich) and flow cytometric analysis of human hematopoietic engraftment was performed as previously described using commercially available mAbs against human leukocyte differentiation antigens to identify engrafted human leukocytes and discriminate their hematopoietic lineages (Chute et al, Blood 100:4433-4439 (2002), Chute et al, Blood 105:576-583 (2005), Trischmann et al, J. Hematother. 2:305-313 (1993)).

### Statistical Analysis and SRC Frequency Measurements

Comparisons of data from in vitro experiments were made using the Student's t test. For purposes of the limiting dilution analysis, a transplanted mouse was scored as positively engrafted if ≧0.1% of the marrow cells expressed human-CD45 via high resolution FACS analysis. This criteria is consistent with previously published criteria for human cell repopulation in NOD/SCID mice (Chute et al, Blood 100:4433-4439 (2002), Dorrell et al, Blood 95:102-110 (2000)). SRC frequency in each cell source was calculated using the maximum likelihood estimator as described previously by Taswell (J. Immunol. 126:1614-1619 (1981), Wang et al, Blood 89:3919-3924 (1997), Ueda et al, J. Clin. Invest. 105:1013-1021 (2000)). Confidence intervals for the frequencies were calculated using the profile likelihood method, and the likelihood ratio test was used to confirm the fit of the model.

### Real Time PCR Analysis of DEAB-Treated HSCs

Extraction of total RNA from Day 0 and Day 7 CB CD34⁺ populations was done using a RNeasy Mini spin column (Qiagen, Valencia, Calif.) according to the manufacturer's recommended protocol. Total RNA isolation from Day 0 CB CD34⁺CD38⁻lin⁻ and the resultant Day 7 progeny was conducted on 1×10⁴ cells/sample, using the RNAqueous-Micro kit (Ambion, Austin, Tex.), using the manufacturer's suggested protocol. Briefly, total RNA was isolated from CD34⁺CD38⁻lin⁻ cells according to the manufacturer's instructions for the RNaqueous®-Micro (Ambion) and reversed-transcribed to cDNA using iScript™ cDNA synthesis Kit (Biorad, Hercules, Calif.). cDNA concentrations were measured with a fluorometer (Turner Designs, Sunnyvale, Calif.) using RiboGreen reagent (Invitrogen). PCR amplification reactions were performed in 13 μl and contained equal amounts of cDNAs, 6.5 μl of iQ SYBR green supermix (Bio-Rad), 0.2 μM of each forward and reverse gene-specific primers for genes of interest and the normalization gene 36B4. PCR was performed on an iCycler (Bio-Rad) according to the following cycling conditions: an initial cycle of 15 min at 95° C.; 45 cycles of 45 sec at 95° C., 15 sec at 55° C., and 15 sec at 72° C.; followed by a melt-curve analysis cycle with steps of 10 sec each at 0.5° C. increments from 60 to 95° C. Amplification rates were visualized and analyzed on ICYCLER IQ optical system software version 3.0 (Bio-Rad). Gene-specific primers were purchased from Integrated DNA Technologies (Coralville, Iowa).

### Analysis of ALDH Activity

Aldehyde dehydrogenase (ALDH) enzyme activity was assayed using the ALDEFLUOR staining kit (Stem Cell Technologies). Cells were suspended at 1×10⁶ cells/ml in Assay Buffer and stained with 200 ng/ml activated ALDEFLUOR reagent and an aliquot was immediately transferred to 6 nM diethylaminobenzaldehyde (DEAB) as a negative control, and the samples were incubated for 30 minutes at 37° C. Following ALDEFLUOR staining, immunophenotype staining was conducted by adding anti-human CD38-PE and anti-human CD34-allophycocyanin (APC) or isotype-matched control antibodies (Becton Dickinson) for 30 minutes on ice. Sample analysis was conducted on a FACScalibur flow cytometer (Becton Dickinson).

### Results

### Inhibition of ALDH Blocks the Differentiation of HSCs in Culture

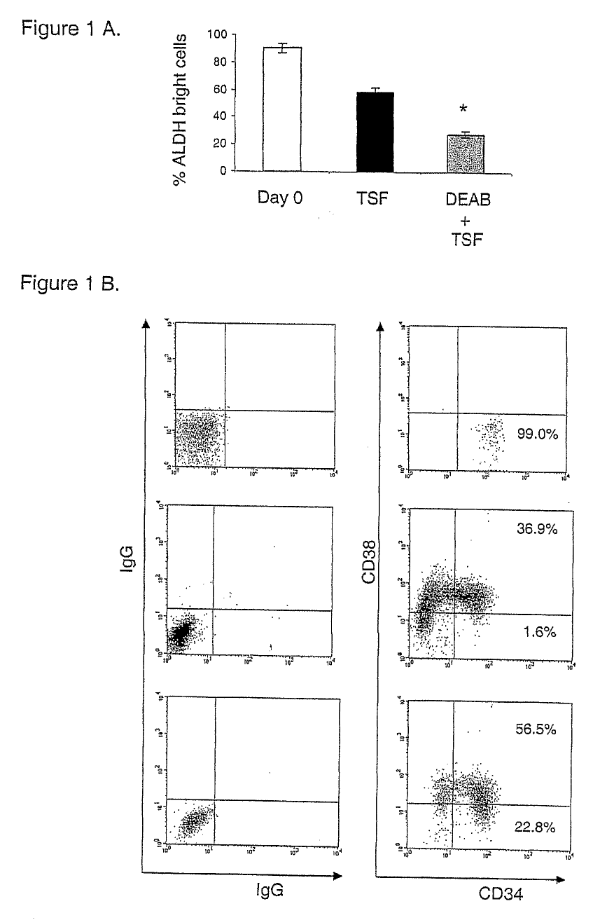

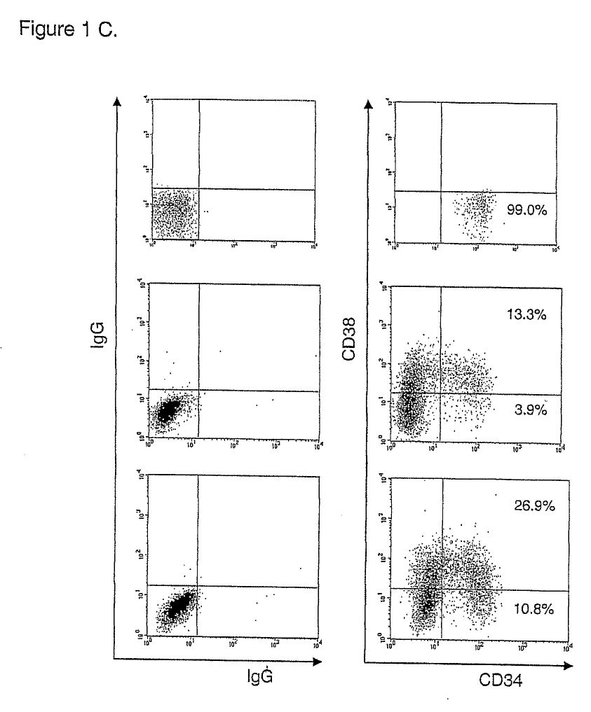

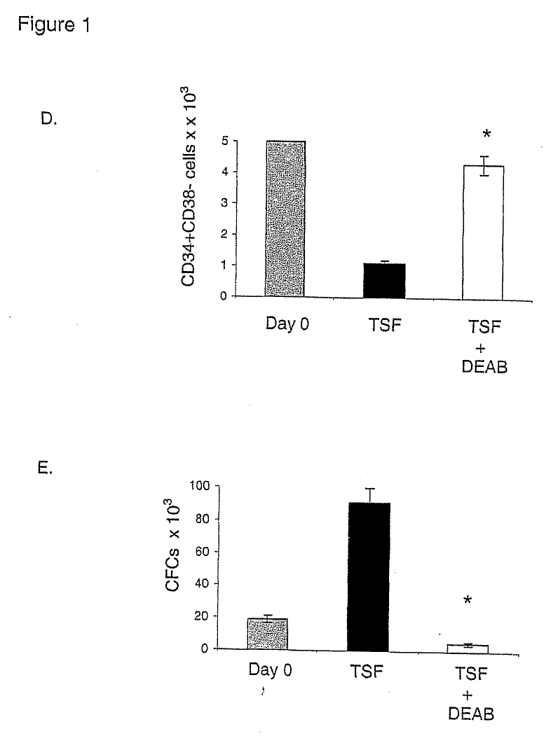

A determination was first made as to whether inhibition of ALDH activity in primary BM and CB CD34⁺CD38⁻lin⁻ cells affected the differentiation of HSCs when cultured in the presence of thrombopoietin 20 ng/mL, SCF 100 ng/mL, and flt-3 ligand 50 ng/mL (TSF) for 7 days. Greater than 90% of day 0 BM CD34⁺CD38⁻lin⁻ cells demonstrated ALDH activity and treatment with 100 μM DEAB+TSF significantly reduced ALDH activity in CD34⁺CD38⁻ cells compared to day 0 or following treatment with TSF alone (FIG. 1A, P=0.001). The progeny of BM and CB CD34⁺CD38⁻lin⁻ cells following culture with DEAB+TSF contained significantly higher percentages of primitive CD34⁺CD38⁻ cells compared to the progeny of TSF alone (FIGS. 1B and 1C, P=0.01, and P=0.02, respectively). DEAB+TSF cultures supported a mean 4-fold total cell expansion and a maintenance of absolute numbers of CD34⁺CD38⁻ cells compared to day 0. Conversely, TSF culture supported a mean 15-fold increase in total cells, but this was associated with a 5-fold decrease in the CD34⁺CD38⁻ cell numbers compared to input (P=0.001, FIG. 1D). As expected, HSC-enriched day 0 BM CD34⁺CD38⁻lin⁻ cells demonstrated little colony forming cell (CFC) content (FIG. 1E). Culture of BM CD34⁺CD38⁻lin⁻ cells with TSF alone caused a 5-fold increase in CFCs compared to input, indicating HSC differentiation during culture. Conversely, the progeny of BM CD34⁺CD38⁻lin⁻ cells cultured with DEAB+TSF contained little CFC content, indicating that DEAB impeded HSC maturation during culture. Morphologic examination of the progeny of DEAB+TSF cultures revealed a predominance of cells with high nuclear:cytoplasmic ratios and prominent nucleoli, whereas TSF-cultured progeny contained primarily bands and myelocytes, suggesting that DEAB treatment maintained more immature progenitors during culture (data not shown). Taken together, these results indicated that inhibition of ALDH activity impeded HSC differentiation in response to cytokines.

### Inhibition of ALDH Activity Increases the Number of Human SRCs in Short-Term Culture

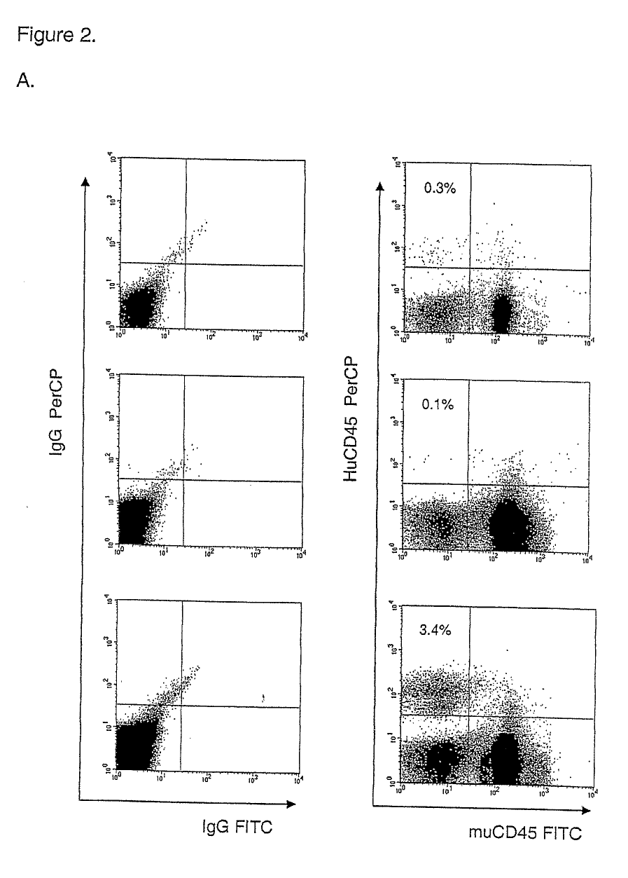

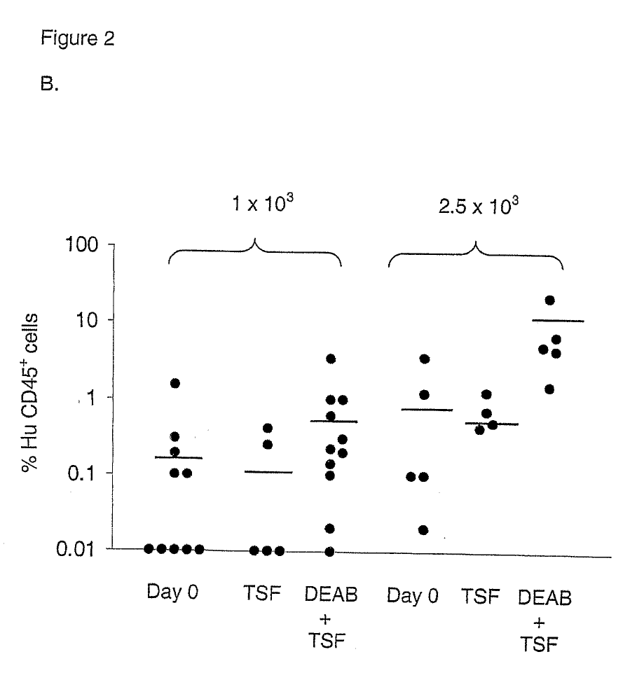

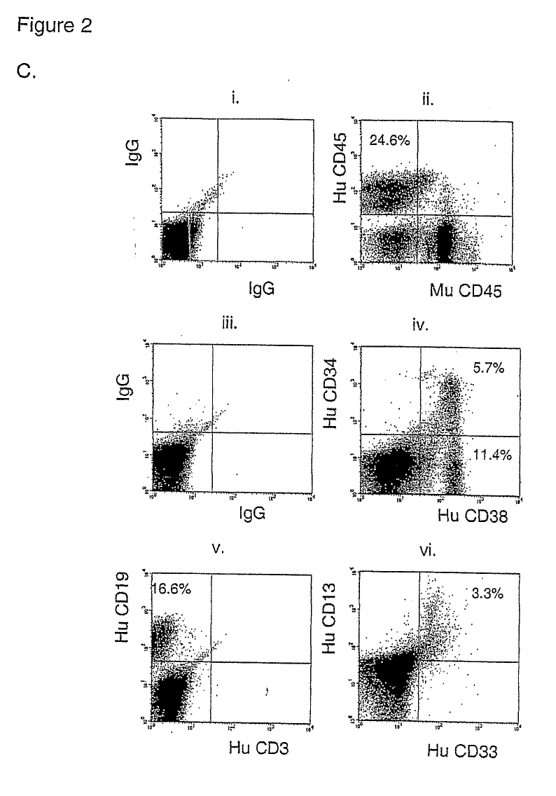

In order to determine whether inhibition of ALDH promoted the self-renewal of HSCs in culture, limiting dilution repopulation assays (Bhatia et al, J. Exp. Med. 186:619-624 (1997)) were performed to estimate the SRC frequency in day 0 CB CD34+CD38−lin− cells or their progeny following culture with DEAB. Analysis of NOD/SCID mice at 8 weeks post-transplant demonstrated that the progeny of CB CD34⁺CD38⁻lin⁻ cells cultured with DEAB+TSF contained significantly increased SRC capacity than either day 0 CB CD34⁺CD38⁻lin⁻ cells or the progeny of cells cultured with TSF alone (FIG. 2A). Over a dose range of 0.5−1×10³, 5 of 16 mice (31%) transplanted with day 0 CB CD34⁺CD38⁻lin⁻ cells and only 2 of 11 mice (18%) transplanted with their progeny following TSF culture demonstrated human CD45⁺ (huCD45⁺) cell engraftment at 8 weeks. Conversely, at the same doses, 9 of 17 mice (53%) transplanted with the progeny of DEAB+TSF cultures showed huCD45⁺ cell repopulation at 8 weeks, with 4-fold higher levels of huCD45⁺ cell engraftment (mean 0.9%, FIG. 2B). At a dose of 2.5×10³, mice transplanted with day 0 CB CD34⁺CD38⁻lin⁻ cells or their progeny following TSF culture again demonstrated only low level huCD45⁺ cell engraftment (mean 0.9% and 0.7%, respectively), whereas mice transplanted with the progeny of DEAB+TSF cultured cells showed 1-log higher levels of huCD45⁺ cell engraftment (mean 8.0%, FIG. 2B). Poisson statistical analysis (Wang et al, Blood 89:3919-3924 (1997), Ueda et al, J. Clin. Invest. 105:1013-1021 (2000)) indicated that the SRC frequency within day 0 CB CD34⁺CD38⁻lin⁻ cells was 1 in 1,700 cells (95% Confidence Interval [CI], 1/930 to 1/3,600). The SRC frequency within the progeny of TSF-cultured CB CD34⁺CD38⁻lin⁻ cells was also 1 in 1,700 cells (CI, 1/800 to 1/4,400). In contrast, the SRC frequency within the progeny of DEAB+TSF-cultured CB CD34⁺CD38⁻lin⁻ cells was 2-fold higher at 1 in 840 cells (CI, 1/490 to 1/1,600). Therefore, inhibition of ALDH not only blocked the differentiation of HSCs but also promoted the amplification of HSCs in culture.

Detailed flow cytometric analysis revealed extensive CD34⁺ progenitor cell, CD19⁺ B lymphoid and CD 33/13⁺ myeloid differentiation in mice transplanted with DEAB+TSF cultured cells, demonstrating that a pluripotent repopulating cell with a normal differentiation program was sustained during culture with DEAB (FIG. 2C).

Treatment with all-Trans Retinoic Acid (ATRA) Promotes the Differentiation of Human HSCs

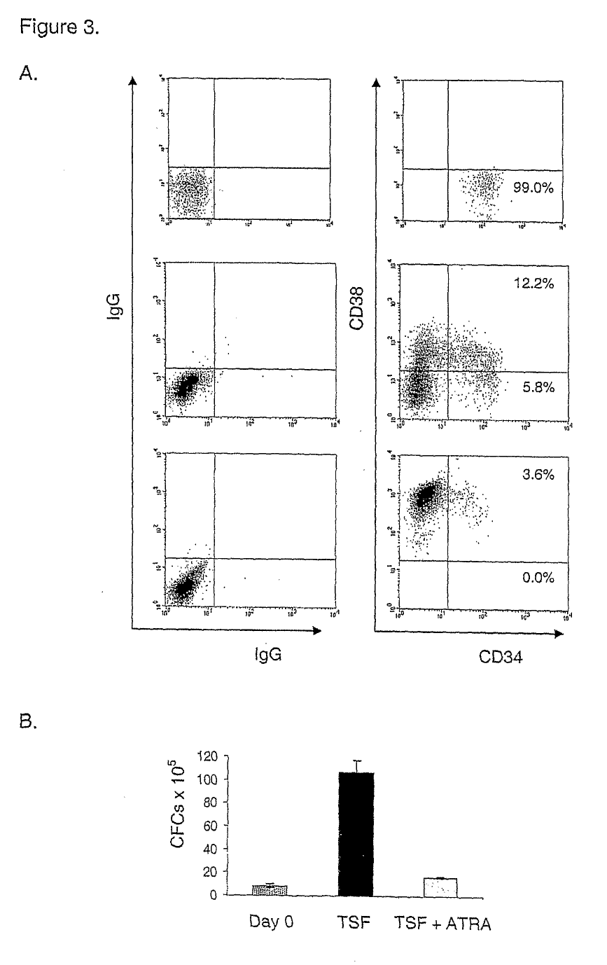

In order to further test the hypothesis that ALDH contributes to HSC differentiation through the production of retinoic acids, an evaluation was made of the effects of ATRA on primary CB CD34⁺CD38⁻lin⁻ cells in culture. When CB CD34⁺CD38⁻lin⁻ cells were cultured with TSF+1 μM ATRA, the percentage of CD34⁺ cells declined relative to TSF alone, and no CD34⁺CD38⁻ cells remained in culture at day 7 (FIG. 3A). The number of CFCs were also decreased in the ATRA+TSF treated cultures compared to cultures with TSF alone, suggesting that ATRA either inhibited CFC formation or promoted terminal differentiation, thereby reducing CFC numbers (FIG. 3B). Taken together, these analyses suggested that ATRA enhanced the differentiation of HSCs during culture with TSF. Moreover, the effects of ATRA on human HSCs were in opposition to the effects of DEAB, suggesting that ALDH may contribute to HSC differentiation through its production of intracellular retinoids. In keeping with this hypothesis, when CB CD34⁺CD38⁻lin⁻ cells were cultured with 1 μM ATRA+100 μM DEAB+TSF, HSC differentiation occurred, indicating that provision of extracellular retinoids overcame DEAB-induced inhibition of intracellular retinoid production (FIG. 6).

Treatment with DEAB Promoted the Expansion of BM HSCs.

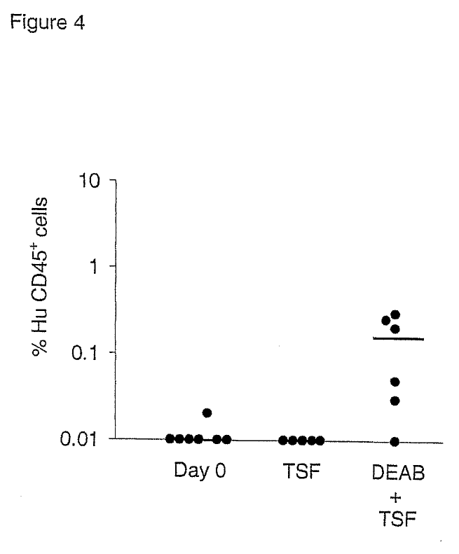

In order to determine whether treatment of DEAB promoted expansion of BM HSCs, NOD/SCID repopulating assays were performed. Over a dose range of 1-2.5×10³ cells, no huCD45⁺ cell repopulation was detected in mice transplanted with either day 0 BM CD34⁺CD38⁻lin⁻ cells or their progeny following culture with TSF alone or TSF+DEAB (n=35 mice). At a dose of 5×10³, neither mice transplanted with day 0 BM CD34⁺CD38⁻lin⁻ cells (0 of 5) nor their progeny following TSF culture demonstrated huCD45⁺ cell repopulation (0 of 5). In contrast, 3 of 6 mice (50%) transplanted with the progeny of DEAB+TSF cultures showed huCD45⁺ cell engraftment (mean 0.1%) (FIG. 4). These results demonstrated that treatment with DEAB promoted the expansion of human BM SRCs. Poisson statistical analysis indicated that the SRC frequency within the DEAB+TSF cultures was 1 in 12,000 (CI, 1/4,700- 1/50,000). Since 0 of 37 mice transplanted with 1-5×10³ day 0 BM CD34⁺CD38⁻lin⁻ cells or TSF-cultured cells demonstrated huCD45⁺ cell engraftment, the SRC frequency in these groups was clearly lower than DEAB-treated cells, but only 1-sided confidence intervals could be developed for these groups, with each having an SRC frequency of < 1/12,000.

### ALDH Inhibition Upregulates HOXB4 in Human HSCs

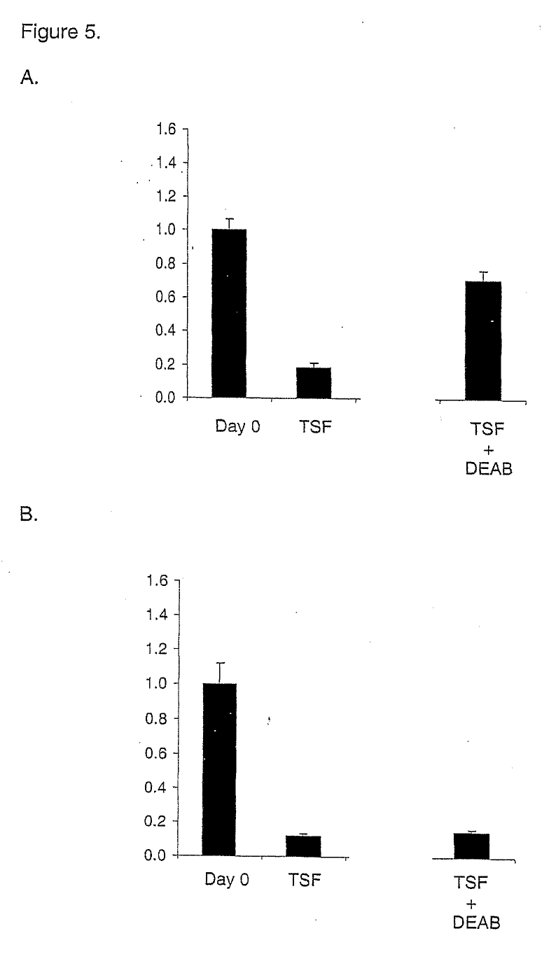

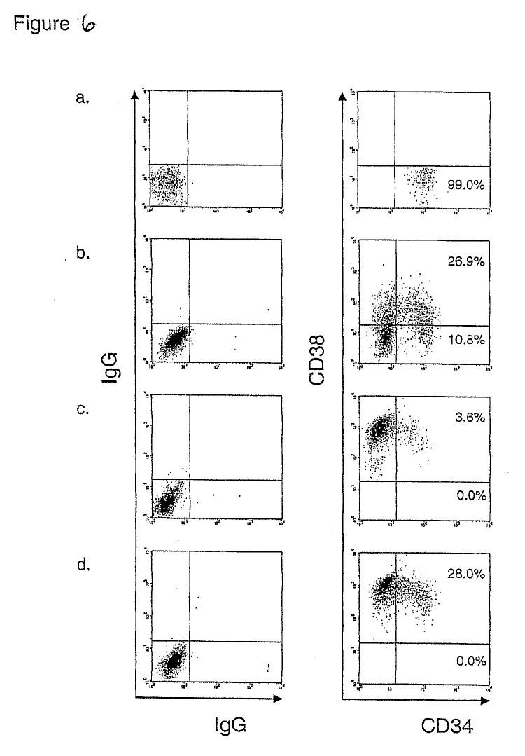

Since HOXB4 and Notch have established roles in HSC self-renewal (Varnum-Finney et al, Nat. Med. 6:1278-1281 (2000), Krosl et al, Nat. Med. 9:1428-1432 (2003)), a determination was made as to whether ALDH inhibition might be regulating HSC self-renewal by altering the transcription of either of these target genes. Interestingly, culture of primary CB CD34⁺CD38⁻lin⁻ cells with TSF alone caused a 5-fold decrease in HOXB4 transcription compared to day 0 CB CD34⁺CD38⁻lin⁻ cells, whereas the addition of DEAB to TSF maintained HOXB4 expression at 80% and 70% of input levels, respectively (FIG. 5). Conversely, treatment with DEAB did not alter Notch transcription compared to TSF alone. Taken together, these data indicate that inhibition of ALDH may promote HSC self-renewal via discrete interactions with other established pathways, such as HOXB4, although the organization of these signals is yet to be elucidated.

Summarizing improved characterization of the pathways that regulate HSC self-renewal will facilitate the development of therapies to amplify HSCs in vitro or in vivo for clinical purposes. In this study, the contributions of the enzyme, ALDH, and retinoid signaling, to human HSC differentiation and self-renewal have been characterized. Aldehyde dehydrogenases are NAD(P)+-dependent enzymes that oxidize a large number of aldehydes to their corresponding carboxylic acids (Bhat et al, Biochem. Pharmacol. 57:195-197 (1999), Haselbeck et al, Dev. Genet. 25:353-364 (1999)). Several different ALDH isoforms (Haselbeck et al, Dev. Genet. 25:353-364 (1999)) have been identified which are responsible for the metabolism of ethanol (Russo et al, Cancer Res. 48:2963-2968 (1988)), catecholamines (Jones et al, Blood 85:2742-2746 (1995)) and the conversion of Vitamin A to its active metabolite, retinoic acid (Bhat et al, Biochem. Pharmacol. 57:195-197 (1999)). ALDH is also a selectable marker of human stern/progenitor cells (Storms et al, Proc. Natl. Acad. Sci. USA 96:9118-9123 (2000), Hess et al, Blood 104:1648-1655 (2004), Storms et al, Blood 106:95-102 (2005)). However, the contribution of ALDH activity to HSC function has remained unknown. In this study, it was shown that competitive inhibition of ALDH blocked the phenotypic and functional maturation of HSCs in response to thrombopoietin, SCF and Flt-3 ligand. ALDH inhibition, coupled with TSF, also gave rise to a 2-fold increase in SRCs and a 9- to 11-fold increase in human hematopoietic cell repopulation in vivo compared to either day 0 CD34⁺CD38⁻lin⁻ cells or their progeny following culture with TSF alone. These data indicate that ALDH is required, at least in part, for HSC differentiation to occur and inhibition of ALDH, when combined with early acting cytokines, is sufficient to induce the amplification of human HSCs. Therefore, these studies demonstrate, for the first time, that ALDH functions to promote the differentiation of human HSCs. Since ALDH-1 activity is required for intracellular retinoic acid production and the addition of ATRA to HSC cultures accelerates HSC differentiation in vitro, it is proposed that ALDH mediates HSC differentiation via the production of retinoic acids.

In light of the observation that inhibition of ALDH promoted HSC expansion, and given that ALDH is required for the intracellular production of retinoic acids, an examination was made of the direct effects of RAR on HSC fate. The results indicated that ATRA, which is an agonist of RAR (Levin et al, Nature 355:359-361 (1992)), induced pronounced differentiation and myeloid maturation of human HSCs by day 7.

The data presented herein demonstrate that modulation of retinoid signaling can induce the expansion of human HSCs. There are several implications of this observations. First, the link between ALDH activity, retinoid signaling and HSC self-renewal has not been previously described and the mechanisms through which ALDH inhibition impedes HSC differentiation is unknown. It is hypothesized that cytokines, such as TSF, induce HSC differentiation via induction of retinoid signaling, perhaps mediated through increased ALDH activity. This hypothesis is supported by the recent observation that another cytokine, IL-3, induces progenitor cell differentiation via activation of Stat5 which, in turn, activates retinoid signaling (Si et al, Blood 100:4401-4409 (2002)). The results also have implications for the development of strategies to amplify human HSCs for clinical purposes. These studies were performed on primary human HSCs and, therefore, the observations are directly translatable to clinical protocols to expand human HSCs. Moreover, in contrast to other reported strategies to expand HSCs in vitro (Reya et al, Nature 423:409-414 (2003), Schiedelmeier et al, Blood 101:1759-1768 (2003)), the targeted approaches described do not depend upon the genetic modification of HSCs or co-culture with surrogate stromal cell niches to achieve potency (Kawano et al, Blood 101:532-540 (2003), Hackney et al, Proc. Natl. Acad. Sci. USA 99:13061-13066 (2002)). Finally, the normal in vivo multilineage differentiation of DEAB-treated HSCs transplanted in NOD/SCID mice demonstrates that ALDH inhibition alters the normal differentiation program of human HSCs. In conclusion, the data indicate that ALDH functions to promote the differentiation of HSCs via production of retinoic acids. Modulation of retinoid signaling via inhibition of ALDH is sufficient to induce the expansion of human HSCs.

All documents and other information sources cited above are hereby incorporated in their entirety by reference.

What is claimed is:

1. A method of promoting expansion of stem cells or progenitor cells, said method comprising contacting said cells an inhibitor of aldehyde dehydrogenase (ALDH) under conditions such that said expansion is effected, wherein said compound is not AGN 194310.

2. The method according to claim 1 wherein cells are hematopoietic stem cells, neuronal stem cells or muscle stem cells.

3. The method according to claim 2 wherein said cells are human hematopoietic stem cells (HSCs).

4. The method according to claim 3 wherein said HSCs are obtained from bone marrow, umbilical cord blood or peripheral blood.

5. The method according to claim 1 wherein said cells are CD34⁺, Thy-1⁺, Lin⁻ stem cells.

6. The method according to claim 1 further comprising contacting said cells with an amount of a hematopoietic growth factor sufficient to effect said expansion.

7. The method according to claim 6 wherein said growth factor is selected from the group consisting of thrombopoietin, SCF and flt-3 ligand.

8. The method according to claim 1 wherein said ALDH inhibitor is DEAB or metabolite thereof.

9. The method according to claim 8, wherein said method further comprises contacting said cells with TSF.

10. A method of identifying a compound that promotes expansion of stem cells or progenitor cells and inhibits differentiation of said cells, said method comprises assaying said compound for its ability to inhibit ALDH, wherein a compound that inhibits ALDH is a candidate compound for promoting expansion of said cells.

11. A method of treating a patient suffering from a disease or disorder associated with decreased levels of myeloid, erythroid, lymphoid or megakaryocyte cells comprising administering to said patient stem cells expanded ex vivo according to the method of claim 1 under conditions such that said treatment is effected.

12. The method according to claim 11 wherein said patient is suffering from leucopenia.

13. A method of accelerating hematopoietic recovery in a patient in need thereof comprising administering to said patient an inhibitor of ALDH in an amount sufficient to effect said acceleration.
